(19) United States
(12) Patent Application Publication
(10) Pub. No.: US 2011/0112164 A1
(43) Pub. Date: May 12, 2011

(54) NOVEL POLYMORPHS AND PROCESSES FOR THEIR PREPARATION

(75) Inventors: Abhay Gaitonde, Maharashtra (IN); Bharati Choudhari, Maharashtra (IN); Prakash Bansode, Maharashtra (IN); Sunanda Phadtare, Phadtare (IN)

(73) Assignees: Generics (UK) Limited, Hertfordshire (GB); Mylan India Private Limited, Maharashtra (IN)

(21) Appl. No.: 12/867,843

(22) PCT Filed: Feb. 20, 2009
(86) PCT No.: PCT/GB09/50170

§ 371 Date: Jan. 4, 2011

(30) Foreign Application Priority Data
Feb. 21, 2008 (IN) 314/KOL/2008

(51) Int. Cl. A61K 31/404; C07D 403/06; A61P 35/00; A61P 35/04

(52) U.S. Cl. 514/414; 548/468

(57) ABSTRACT

The present invention relates to novel polymorph forms III and IV of sunitinib malate, pharmaceutical compositions comprising the novel polymorphs and the use of the pharmaceutical compositions. The present invention further relates to processes for the preparation of polymorph form I, III and IV of sunitinib malate.

### CROSS-REFERENCE TO RELATED APPLICATION(S)

This application is a Section 371 National Stage Application of International No. PCT/GB2009/050170, filed 20 Feb. 2009 and published as WO 2009/104021 A2 on 27 Aug. 2009, which claims priority from the India Application 314/KOL/2008, filed 21 Feb. 2008, the contents of which are incorporated herein in their entirety for all purposes.

### FIELD OF THE INVENTION

The present invention relates to novel polymorph forms III and IV of sunitinib malate, pharmaceutical compositions comprising the novel polymorphs and the use of the pharmaceutical compositions. The present invention further relates to processes for the preparation of polymorph form I, III and IV of sunitinib malate.

### BACKGROUND OF THE INVENTION

Sunitinib malate, represented by formula (I) and chemically named (Z)—N-[2-(diethylamino)ethyl]-5-(5-fluoro-2-oxo-2,3-dihydro-1H-indole-3-ylidenemethyl)-2,4-dimethyl-1H-pyrrole-3-carboxamide 2(S)-hydroxybutanedioic acid, is a tyrosine kinase inhibitor (TKI) that targets and blocks the signaling pathways of multiple selected receptor tyrosine kinases (RTKs). Through competitive inhibition of ATP binding sites, sunitinib malate inhibits the TK activity of a group of closely related RTKs, all of which are involved in various human malignancies: the vascular endothelial growth factor receptors (VEGFR-1, -2, -3), the platelet derived growth factor receptors (PDGF-R), the stem cell factor (KIT), CSF-1R, Flt3, and RET. Sunitinib malate is therefore useful for the treatment of cancer and tumours. It is currently marketed for the treatment of unresectable and/or metastatic malignant gastrointestinal stromal tumour (GIST) and advanced and/or metastatic renal cell carcinoma (MRCC).

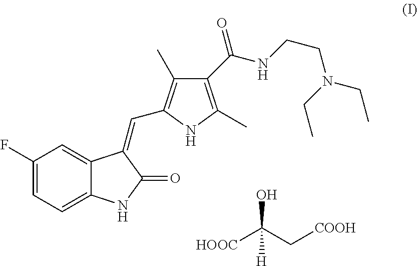

Polymorphs are distinct solids sharing the same molecular formula, yet each polymorph may have distinct physical properties. Therefore a single compound may give rise to a variety of polymorphic forms where each form has different and distinct physical properties, such as different solubility profiles, different melting point temperatures and/or different X-ray diffraction peaks. The solubility of each polymorph may vary and consequently identifying the existence of polymorphs of an active pharmaceutical ingredient (API) is essential for providing pharmaceutical compositions with predictable solubility profiles. It is desirable to investigate all solid state forms of a drug, including all polymorphic forms. Polymorphic forms of a compound can be distinguished in a laboratory by X-ray diffraction spectroscopy and by other methods such as infrared spectrometry. Additionally, the properties of polymorphic forms of the same active pharmaceutical ingredient are well known in the pharmaceutical art to have an effect on the manufacture of drug product compositions comprising the API. For example, the solubility, stability, flowability, tractability and compressibility of the API as well as the safety and efficacy of drug product can be dependent on the crystalline or polymorphic form.

Sunitinib malate was first described in U.S. Pat. No. 6,573,293. Processes for the synthesis of sunitinib are also described in the prior art. The prior art also describes the L-malate salt of sunitinib.

The discovery of new polymorphic forms of a pharmaceutically useful compound provides a new opportunity to improve the performance characteristics of a pharmaceutical product. It also adds to the material that a formulation scientist has available for designing, for example, a pharmaceutical dosage form of a drug with a targeted release profile or other desired characteristic.

Crystal polymorphic forms I and II of sunitinib malate and methods of preparing the crystals are disclosed in prior art patent application WO 03/016305. However, there are serious disadvantages in these forms and/or the methods to prepare them. Form II is hygroscopic, thermodynamically unstable and appears to readily convert to form I. Form I was obtained by slurry formation in acetonitrile. Alternatively, form I was prepared by slurry formation from form II in acetonitrile.

Slurry formation is not a favourable method of producing crystalline material on a commercial scale as the solid does not completely dissolve in the solvent, as a result of which it is difficult to produce consistent and reproducible products. It is also difficult to produce chemically and polymorphically pure products from slurries. In contrast, preparation of crystals from solutions, where there is no slurry formation, typically leads to more reproducible results and purer products, particularly on a commercial production scale.

Therefore a need for a solution exists that overcomes one or more of the above-identified disadvantages.

### DEFINITIONS

As used herein, the term “sunitinib malate” refers to sunitinib (S)-malate.

As used herein, the terms “crystalline form”, “polymorph”, “polymorph form” and “polymorphic form” are used interchangeably.

The terms “X-ray diffraction pattern” and “XRD spectrum” are used interchangeably herein and preferably refer to an X-ray powder diffraction (XRPD) pattern or spectrum.

As used herein, the term “ambient temperature” refers to a temperature range from about 15° C. to about 30° C., preferably from about 22° C. to about 27° C.

As used herein, crystalline form I of sunitinib malate is as defined in WO 03/016305, i.e. characterized by an X-ray diffraction pattern having peaks at 2θ values at about 13.2, 19.4, 24.2 and 25.5 °2θ.

The following solvent acronyms are used:
DCM dichloromethane
DEE diethyl ether

### DMAc N,N-dimethylacetamide

### DMF N,N-dimethylformamide

DMSO dimethylsulfoxide
EAA ethyl acetoacetate
IPA iso-propanol
MEK methyl ethyl ketone
MIBK methyl iso-butyl ketone
TBME t-butyl methyl ether
THF tetrahydrofuran

### SUMMARY OF THE INVENTION

The present invention provides novel polymorph form III and form IV of sunitinib malate, which are crystalline, non-hygroscopic and stable.

In one aspect, the invention provides novel polymorphs of sunitinib malate with improved properties and processes to produce them.

In another aspect, the invention provides an improved process for the preparation of form I of sunitinib malate which avoids a slurry preparation.

It still another aspect, the present invention provides pharmaceutical compositions containing the polymorphs described herein.

According to a first aspect of the present invention there is provided a crystalline form III of sunitinib malate with a characteristic XRD spectrum having three or more peaks (preferably four or more, five or more, six or more, or seven peaks) with 2θ values selected from 4.05, 8.02, 9.13, 10.44, 12.01, 16.00 and 17.80±0.2 °2θ. Preferably the crystalline form III of sunitinib malate has a characteristic XRD spectrum having major peaks with 2θ values at 4.05, 8.02, 9.13, 10.44, 12.01, 16.00 and 17.80.

The crystalline form III of sunitinib malate according to the first aspect of the invention is further characterized by a differential scanning calorimetry (DSC) with an endothermic peak at about 227° C. (preferably about 227.28° C.); a capillary melting point of approximately 216° C.; and a thermo-gravimetric analysis (TGA) loss of about 0.29%. The crystalline form III of sunitinib malate according to the first aspect of the invention is non-hygroscopic and stable.

According to a second aspect of the present invention there is provided a process for the preparation of crystalline form III of sunitinib malate, comprising the steps of:

(a) dissolving or suspending sunitinib malate, or sunitinib and malic acid, in a solvent;
(b) cooling the solution or suspension obtained in step (a);
(c) isolating the crystalline solid obtained in step (b); and
(d) drying the solid obtained in step (c).

In step (a) preferably sunitinib malate, or sunitinib and malic acid, is/are dissolved, preferably sunitinib malate is dissolved. The solvent in step (a) is preferably a non-hydroxylic solvent, such as an ester. A preferred ester is ethyl acetoacetate. Preferably, the solvent in step (a) is heated to dissolve the sunitinib malate. The solvent is preferably heated at the reflux temperature of the solvent, preferably between 110-115° C. Preferably, step (b) comprises cooling to ambient temperature.

According to a third aspect of the present invention there is provided a process for the preparation of crystalline form III of sunitinib malate, comprising the steps of:

(a) dissolving or suspending sunitinib malate, or sunitinib and malic acid, in a solvent;
(b) adding an anti-solvent to the solution or suspension obtained in step (a);
(c) cooling the solution or suspension obtained in step (b);
(d) isolating the crystalline solid obtained in step (c); and
(e) drying the solid obtained in step (d).

In step (a) preferably sunitinib malate, or sunitinib and malic acid, is/are dissolved, preferably sunitinib malate is dissolved. Preferably, the solvent in step (a) is a non-hydroxylic solvent, such as an ester. A preferred ester is ethyl acetoacetate. The solvent in step (a) is preferably heated, typically at reflux temperature. Preferably, the reflux temperature is between 110-115° C. Preferably, step (c) comprises cooling to ambient temperature.

The anti-solvent used in step (b) of the third aspect of the invention is preferably a non-hydroxylic solvent, such as an ester, a ketone or a hydrocarbon. The anti-solvent is preferably an ester, most preferably iso-butyl acetate.

According to a fourth aspect of the present invention there is provided a crystalline form IV of sunitinib malate characterized by an X-ray diffraction pattern having three or more peaks (preferably four or more, five or more, six or more, seven or more, eight or more, nine or more, ten or more, eleven or more, or twelve peaks) at 2θ values selected from 8.69, 13.01, 19.40, 20.32, 21.80, 24.18, 25.49, 26.13, 27.04, 28.23, 31.10 and 32.93±0.2 °2θ. Preferably the crystalline form IV of sunitinib malate is characterized by an X-ray diffraction pattern having peaks at 2θ values at 8.69, 13.01, 19.40, 20.32, 21.80, 24.18, 25.49, 26.13, 27.04, 28.23, 31.10 and 32.93.

The crystalline form IV of sunitinib malate according to the fourth aspect of the invention is further characterized by a differential scanning calorimetry (DSC) with an endothermic peak at about 204° C. (preferably about 204.03° C.); a capillary melting point of approximately 198° C.; and a thermo-gravimetric analysis (TGA) loss of about 0%. The crystalline form IV of sunitinib malate according to the fourth aspect of the invention is non-hygroscopic and stable.

According to a fifth aspect of the present invention there is provided a process for the preparation of crystalline form IV of sunitinib malate, comprising the steps of:

(a) dissolving or suspending sunitinib malate, or sunitinib and malic acid, in a solvent;
(b) cooling the solution or suspension obtained in step (a);
(c) isolating the crystalline solid obtained in step (b); and
(d) drying the solid obtained in step (c).

In step (a) preferably sunitinib malate, or sunitinib and malic acid, is/are dissolved, preferably sunitinib malate is dissolved. Preferably, the solvent in step (a) is water. Typically, the solvent in step (a) is heated to dissolve the sunitinib malate. Preferably, the solvent in step (a) is heated at 60-80° C., most preferably at approximately 62° C. Preferably, step (b) comprises cooling to ambient temperature.

According to a sixth aspect of the present invention there is provided a process for the preparation of crystalline form IV of sunitinib malate, comprising the steps of:

(a) dissolving or suspending sunitinib malate, or sunitinib and malic acid, in a solvent;
(b) adding an anti-solvent to the solution or suspension obtained in step (a);
(c) cooling the solution or suspension obtained in step (b);
(d) isolating the crystalline solid obtained in step (c); and
(e) drying the solid obtained in step (d).

In step (a) preferably sunitinib malate, or sunitinib and malic acid, is/are dissolved, preferably sunitinib malate is dissolved. Preferably, the solvent in step (a) is water. Preferably, the solvent in step (a) is heated at 60-80° C., most preferably at approximately 75° C. Preferably, step (c) comprises cooling to ambient temperature.

Preferably the anti-solvent for the sixth aspect of the invention is selected from an alcohol, a ketone, an ester, a nitrile, an ether, a hydrocarbon or a halogenated hydrocarbon. More preferably, the anti-solvent is selected from an alcohol, acetonitrile, acetone, 1,4-dioxane or THF, more preferably the anti-solvent is selected from an alcohol, acetonitrile, acetone or 1,4-dioxane. Preferably, the anti-solvent is an alcohol, such as a C1 to C6 alcohol, or a substituted alcohol, such as ethoxy ethanol. Most preferably the alcohol is selected from methanol, ethanol, n-propanol, iso-propanol or t-butanol.

According to a seventh aspect of the present invention there is provided a process for the preparation of crystalline form I of sunitinib malate, comprising the steps of:

(a) dissolving or suspending sunitinib malate, or sunitinib and malic acid, in a solvent;
(b) cooling the solution or suspension obtained in step (a);
(c) isolating the crystalline solid obtained in step (b); and
(d) drying the solid obtained in step (c).

In step (a) preferably sunitinib malate, or sunitinib and malic acid, is/are dissolved, preferably sunitinib malate is dissolved. Preferably, the solvent in step (a) is a hydroxylic solvent or a polar aprotic solvent, which is preferably selected from cyclopentanol, cyclohexanol, methoxy ethanol or N,N-dimethylacetamide. Preferably, the solvent in step (a) is heated to dissolve the sunitinib malate, preferably to 99-122° C. Preferably, step (b) comprises cooling to ambient temperature.

According to an eighth aspect of the present invention there is provided a process for the preparation of crystalline form I of sunitinib malate, comprising the steps of:

(a) dissolving or suspending sunitinib malate, or sunitinib and malic acid, in a solvent;
(b) adding an anti-solvent to the solution or suspension obtained in step (a);
(c) cooling the solution or suspension obtained in step (b);
(d) isolating the crystalline solid obtained in step (c); and
(e) drying the solid obtained in step (d).

In step (a) preferably sunitinib malate, or sunitinib and malic acid, is/are dissolved, preferably sunitinib malate is dissolved. Preferably, the solvent in step (a) is a polar aprotic solvent, an alcohol or an alkoxy alcohol. Preferably, the polar aprotic solvent is DMF, DMAc or DMSO, and preferably the alkoxy alcohol is methoxy ethanol. Typically, the solvent in step (a) is heated to dissolve the sunitinib malate. Preferably, the solvent is heated between 55-115° C. Preferably, step (c) comprises cooling to ambient temperature.

The anti-solvent for the eighth aspect of the invention is preferably selected from an alcohol, a ketone, an ester, a nitrile, an ether, a hydrocarbon or a halogenated hydrocarbon. Preferably, the anti-solvent is selected from water, methanol, ethanol, 1-propanol, 1-butanol, 1-pentanol, iso-propanol, iso-butanol, t-butanol, ethoxy ethanol, acetonitrile, acetone, methyl ethyl ketone, methyl iso-butyl ketone, diethyl ketone, ethyl acetate, iso-propyl acetate, iso-butyl acetate, n-pentyl acetate, DCM, 1,4-dioxane, THF, t-butyl methyl ether, diethyl ether, toluene or xylene.

According to a ninth aspect of the present invention there is provided a crystalline form I of sunitinib malate obtained by a process according to the seventh or eighth aspect of the present invention.

The crystalline forms of sunitinib malate of the present invention may exist in one or more tautomeric, hydrate and/or solvate forms. The present invention embraces all tautomeric forms and their mixtures, all hydrate forms and their mixtures, and all solvate forms and their mixtures.

Preferably the crystalline forms of sunitinib malate according to the above described aspects and embodiments have a chemical purity of greater than 95%, 96%, 97%, 98% or 99% (as measured by HPLC). Preferably the crystalline forms of sunitinib malate according to the above described aspects and embodiments have a polymorphic purity of greater than 95%, 96%, 97%, 98% or 99% (as measured by XRPD or DSC).

In a further embodiment of the processes of the present invention, the crystalline forms of sunitinib malate are obtained on an industrial scale, preferably in batches of 0.5 kg, 1 kg, 5 kg, 10 kg, 50 kg, 100 kg, 500 kg or more.

According to a tenth aspect of the present invention there is provided a pharmaceutical composition comprising sunitinib malate form III or form IV, or sunitinib malate form I obtained by a process according to the seventh or eighth aspect of the invention. Preferably, the pharmaceutical composition according to the tenth aspect of the invention is for use in the treatment of cancer. Preferably, the use is the treatment of cancer and tumours. More preferably, the use is the treatment of unresectable and/or metastatic malignant gastrointestinal stromal tumour (GIST) or advanced and/or metastatic renal cell carcinoma (MRCC).

Preferably the sunitinib malate form III according to the first aspect of the present invention, the sunitinib malate form IV according to the fourth aspect of the present invention, and the sunitinib malate form I according to the ninth aspect of the present invention, are suitable for use in medicine, preferably for treating or preventing cancer or a tumour, preferably for treating or preventing unresectable and/or metastatic malignant gastrointestinal stromal tumour (GIST) or advanced and/or metastatic renal cell carcinoma (MRCC).

According to an eleventh aspect of the present invention there is provided a use of sunitinib malate form III according to the first aspect of the present invention, or sunitinib malate form IV according to the fourth aspect of the present invention, or sunitinib malate form I according to the ninth aspect of the present invention, in the manufacture of a medicament for treating or preventing cancer or a tumour, preferably for treating or preventing unresectable and/or metastatic malignant gastrointestinal stromal tumour (GIST) or advanced and/or metastatic renal cell carcinoma (MRCC).

According to a twelfth aspect of the present invention there is provided a method of treating or preventing cancer or a tumour, the method comprising administering to a patient in need thereof a therapeutically of prophylactically effective amount of sunitinib malate form III according to the first aspect of the present invention, or sunitinib malate form IV according to the fourth aspect of the present invention, or sunitinib malate form I according to the ninth aspect of the present invention. Preferably the method is for treating or preventing unresectable and/or metastatic malignant gastrointestinal stromal tumour (GIST) or advanced and/or metastatic renal cell carcinoma (MRCC). Preferably the patient is a mammal, preferably a human.

### BRIEF DESCRIPTION OF THE ACCOMPANYING FIGURES

FIG. 1 describes the X-ray powder diffraction (XRPD) of sunitinib malate form III.

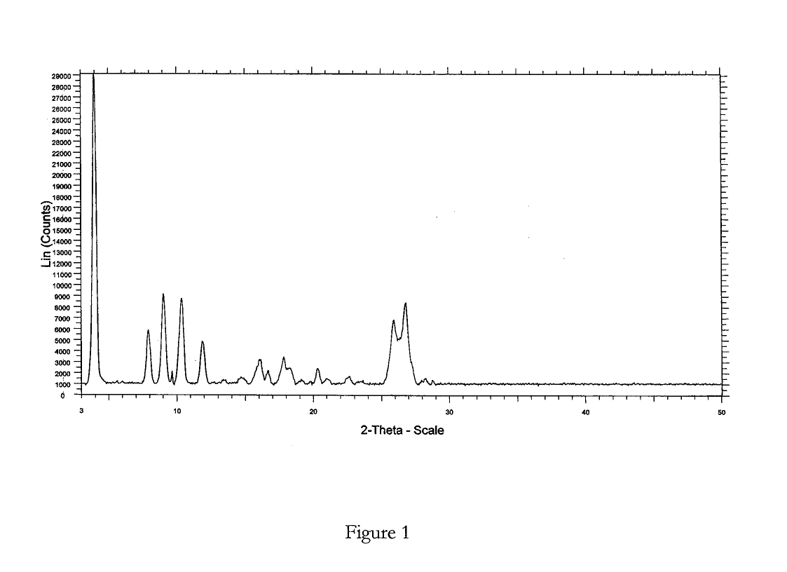

FIG. 2 describes the differential scanning calorimetry (DSC) of sunitinib malate form III.

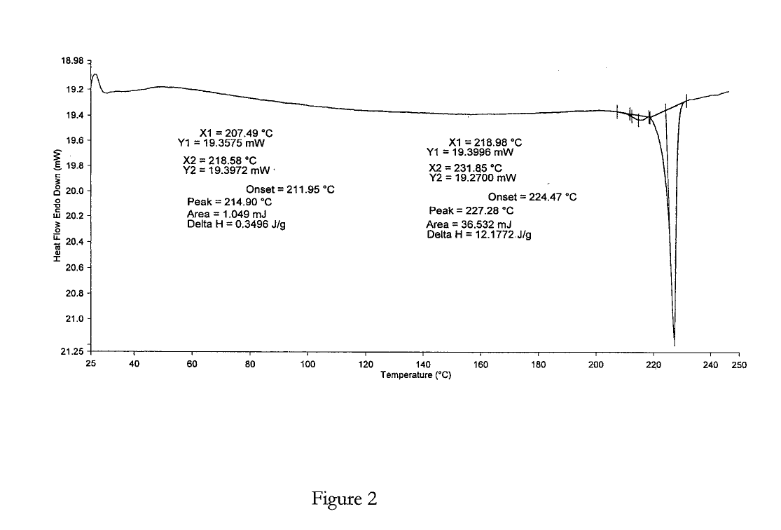

FIG. 3 describes the thermo-gravimetric analysis (TGA) of sunitinib malate form III.

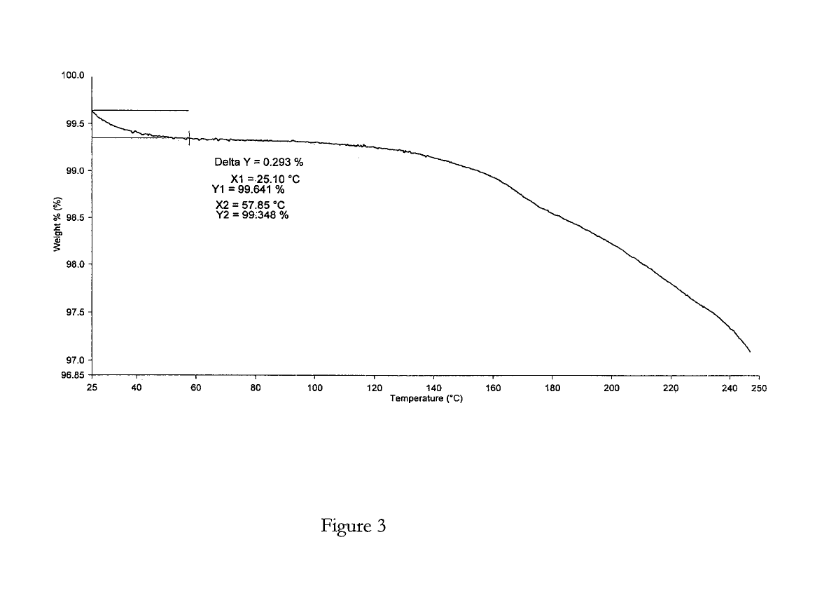

FIG. 4 describes the X-ray powder diffraction (XRPD) of sunitinib malate form IV.

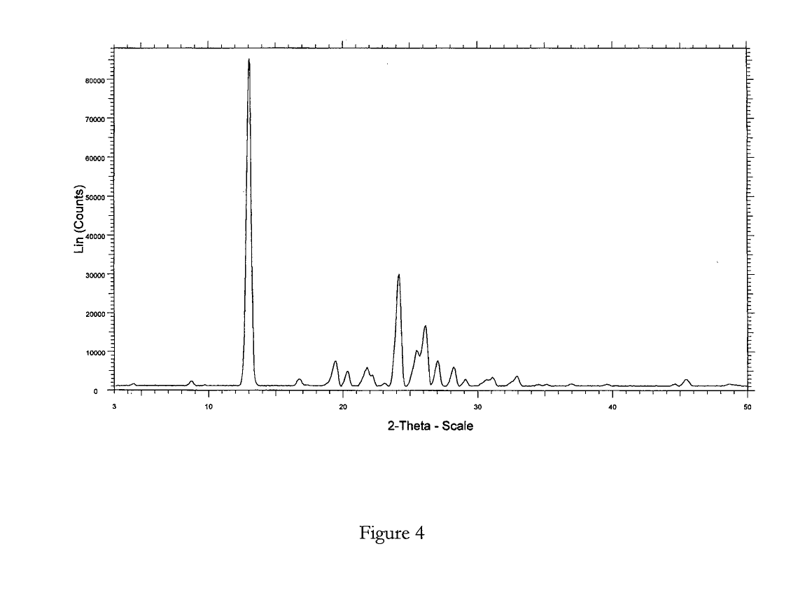

FIG. 5 describes the differential scanning calorimetry (DSC) of sunitinib malate form IV.

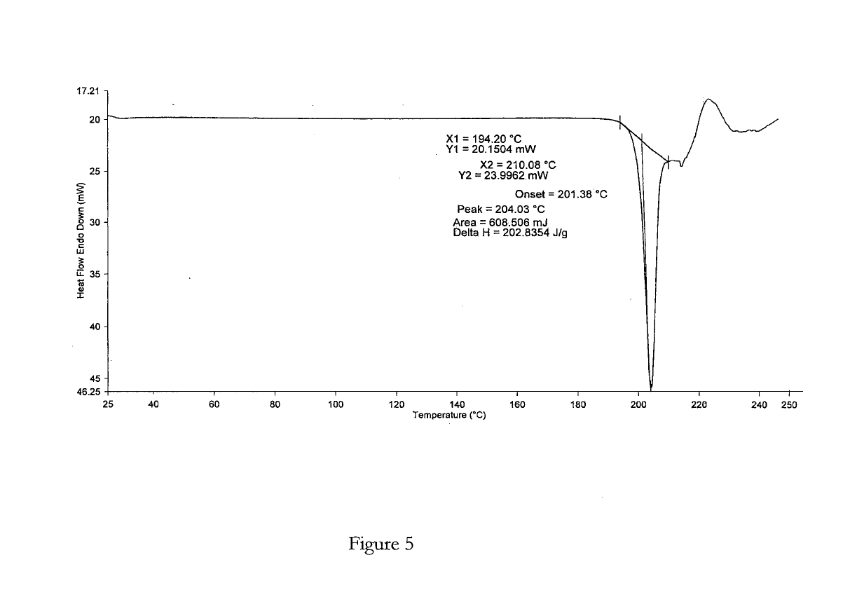

FIG. 6 describes the thermo-gravimetric analysis (TGA) of sunitinib malate form IV.

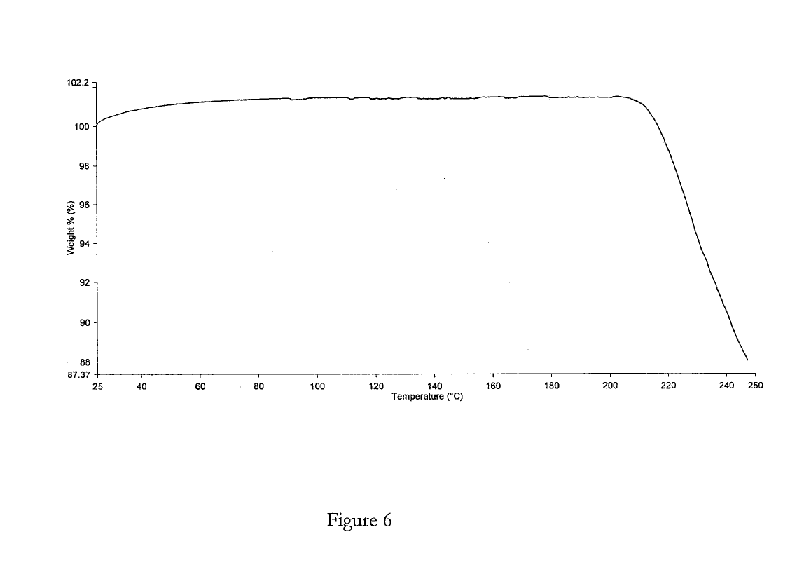

### DETAILED DESCRIPTION OF THE INVENTION

As outlined above, the present invention provides two new crystalline forms of sunitinib malate, form III and form IV, which are non-hygroscopic, polymorphically stable and have beneficial properties which avoid the problems associated with prior art forms.

In addition, convenient processes for the preparation of forms III and IV have been provided and preferred embodiments of these processes are described below.

A preferred embodiment of the process for the preparation of crystalline form III of sunitinib malate comprises the steps of:

(a) dissolving sunitinib malate in ethyl acetoacetate at reflux temperature, preferably at 110-115° C.;
(b) cooling the solution obtained in step (a);
(c) filtering the suspension obtained in step (b) to isolate the novel polymorph; and
(d) drying the solid obtained in step (c).

In a preferred process, in step (a) a clear solution is obtained by dissolving sunitinib malate in ethyl acetoacetate at reflux temperature, preferably at 110-115° C. Preferably, the solution obtained in step (a) is cooled to a temperature of 22-27° C. Preferably, in step (c) the novel polymorph is isolated by filtration under vacuum. Preferably, in step (d) the solid is dried under vacuum at about 40° C.

Another preferred embodiment of the process for the preparation of sunitinib malate form III comprises the steps of:

(a) dissolving sunitinib malate in ethyl acetoacetate at 110-115° C.;
(b) adding iso-butyl acetate to the solution obtained in step (a);
(c) cooling the solution obtained in step (b);
(d) isolating the crystalline solid obtained in step (c), followed by drying to obtain sunitinib malate form III.

A preferred embodiment of the process for the preparation of crystalline form IV of sunitinib malate comprises the steps of:

(a) dissolving sunitinib malate in water at elevated temperature, preferably about 62° C.;
(b) cooling the solution obtained in step (a) to ambient temperature;
(c) filtering the suspension obtained in step (b) to isolate the novel polymorph; and
(d) drying the solid obtained in step (c).

The present invention also provides a novel process for the preparation of sunitinib malate form IV comprising the steps of:

(a) dissolving sunitinib malate in water at elevated temperature, preferably about 62° C.;
(b) adding an anti-solvent to the solution obtained in step (a) at the same elevated temperature, preferably about 62° C.;
(c) cooling the solution obtained in step (b);
(d) isolating the crystalline solid obtained in step (c); and
(e) drying the solid obtained in step (d).

The anti-solvent used is preferably an alcohol, a ketone, an ester, a nitrile, an ether, a hydrocarbon or a halogenated hydrocarbon. Preferably, in step (c) the solution is cooled to a temperature of 22-27° C. Preferably, in step (d) the solid is isolated by filtration under vacuum. Preferably, in step (e) the solid is dried under vacuum at about 40° C.

According to a further preferred embodiment of the present invention there is provided a process for the preparation of sunitinib malate form IV, comprising the steps of:

(a) dissolving sunitinib malate in water at about 75° C.;
(b) adding an anti-solvent to the solution obtained in step (a);
(c) cooling the solution obtained in step (b); and
(d) isolating the crystalline solid obtained in step (c).

The present invention also provides improved methods of producing crystalline form I of sunitinib malate on a commercial scale with consistent and reproducible products. The improved process to produce form I provides chemically and polymorphically pure products from solutions. Preferred embodiments of the process are further described below.

According to a preferred embodiment of the invention, there is provided a process for preparing form I of sunitinib malate, comprising the steps of:

(a) dissolving or suspending sunitinib malate in an organic solvent at reflux temperature;
(b) cooling the solution or suspension obtained in step (a) to ambient temperature;
(c) filtering the suspension obtained in step (b) to isolate the novel polymorph; and
(d) drying the solid obtained in step (c).

In a preferred embodiment of this process, the organic solvent(s) in step (a) is/are chosen from the group comprising lower and higher alcohols or hydrocarbons. Preferably, the organic solvent is heated until at least 80%, preferably 90% and most preferably about 100% of the sunitinib malate is dissolved in the organic solvent. In a preferred embodiment, the sunitinib malate is dissolved in the organic solvent by heating said organic solvent to a temperature that facilitates the sunitinib malate dissolving or by other means such as sonication to facilitate dissolution. Optionally, the solution in step (a) is filtered. Preferably, in step (c) the crystalline solid is isolated by filtration. In step (d) preferably the crystalline solid is dried, most preferably under vacuum.

According to another preferred embodiment of the invention, there is provided a novel process for the preparation of sunitinib malate form I, comprising the steps of:

(a) dissolving sunitinib malate in an organic solvent at elevated temperature, preferably 55-115° C.;
(b) adding an anti-solvent to the solution obtained in step (a);
(c) cooling the solution obtained in step (b);
(d) isolating the crystalline solid obtained in step (c), followed by drying to obtain sunitinib malate form I.

A preferred embodiment of the process for the preparation of sunitinib malate form I comprises the steps of:

(a) dissolving sunitinib malate in an organic solvent at elevated temperature;
(b) adding an anti-solvent to the solution obtained in step (a) at the same elevated temperature;
(c) cooling the solution obtained in step (b);
(d) isolating the crystalline solid obtained in step (c); and
(e) drying the solid obtained in step (d).

In another embodiment of the process to prepare form I, the solution is obtained by dissolving sunitinib malate in DMF at elevated temperature, preferably at 55-115° C. The temperature employed is preferably about 80° C.

In another embodiment of the process to prepare form I, the solution is obtained by dissolving sunitinib malate in DMSO at elevated temperature, preferably at 55-115° C. The temperature employed is preferably about 55° C.

In another embodiment of the process to prepare form I, the solution is obtained by dissolving sunitinib malate in methoxy ethanol at elevated temperature, preferably at 55-115° C. The temperature employed is preferably about 115° C.

In yet another embodiment of the process to prepare form I, the anti-solvent is added to the solution of sunitinib malate in an organic solvent at a respective elevated temperature, preferably at 55-115° C.

In yet another embodiment of the process to prepare form I, the anti-solvent used is selected from an alcohol, a ketone, an ester, a nitrile, an ether, a hydrocarbon and a halogenated hydrocarbon.

In another embodiment of the process to prepare form I, the solution from step (b) is cooled to a temperature of 22-27° C. In another embodiment, in step (d) the solid is isolated by filtration under vacuum. In a further embodiment, in step (e) the solid is dried under vacuum at about 40° C.

A summary of the solvents used for the preparation of crystalline forms I, III and IV of sunitinib malate are summarized in Tables 1 to 8 below.

### Form I

### A. Single Solvent: Solutions

TABLE 1

| | Amount | Temperature | Yield |
|---|---|---|---|
| Solvent | (vol) | (° C.) | (% w/w) |
| Cyclopentanol | 25 | 100 | 69 |
| Cyclohexanol | 25 | 110 | 98 |
| Methoxy Ethanol | 25 | 122 | 60 |
| DMAc | 25 | 99 | 77 |

### B. Combination of Solvents: Solutions

i. DMF Combinations:

TABLE 2

| | Amount | Temperature | Yield |
|---|---|---|---|
| Solvent/Anti-solvent | (vol) | (° C.) | (% w/w) |
| DMF/Water | 3/5 | 80 | 92 |
| DMF/Methanol | 3/5 | 80 | 86 |
| DMF/Ethanol | 3/5 | 80 | 78 |
| DMF/1-Propanol | 3/10 | 80 | 81 |
| DMF/1-Butanol | 3/5 | 80 | 81 |
| DMF/1-Pentanol | 3/5 | 80 | 92 |
| DMF/iso-Propanol | 3/5 | 80 | 88 |
| DMF/iso-Butanol | 3/5 | 80 | 96 |
| DMF/t-Butanol | 3/5 | 80 | 89 |
| DMF/Ethoxy Ethanol | 3/5 | 80 | 80 |
| DMF/Acetonitrile | 3/5 | 80 | 90 |
| DMF/Acetone | 3/5 | 80 | 93 |
| DMF/Methyl Ethyl Ketone | 3/5 | 80 | 98 |
| DMF/MIBK | 3/5 | 80 | 92 |
| DMF/Diethyl Ketone | 3/5 | 80 | 75 |
| DMF/Ethyl Acetate | 3/5 | 80 | 86 |
| DMF/iso-Propyl Acetate | 3/5 | 80 | 88 |
| DMF/iso-Butyl Acetate | 3/5 | 80 | 84 |
| DMF/n-Pentyl Acetate | 3/5 | 80 | 92 |
| DMF/DCM | 3/5 | 80 | 99 |
| DMF/1,4-Dioxane | 3/5 | 80 | 94 |
| DMF/THF | 3/5 | 80 | 87 |
| DMF/t-Butyl Methyl Ether | 3/5 | 80 | 87 |
| DMF/Diethyl Ether | 3/5 | 80 | 97 |
| DMF/Toluene | 3/5 | 80 | 95 |
| DMF/Xylene | 3/5 | 80 | 92 |

ii. DMSO Combinations:

TABLE 3

| | Amount | Temperature | Yield |
|---|---|---|---|
| Solvent/Anti-solvent | (vol) | (° C.) | (% w/w) |
| DMSO/Water | 3/30 | 55 | 80 |
| DMSO/Methanol | 3/5 | 55 | 97 |
| DMSO/Ethanol | 3/5 | 55 | 85 |
| DMSO/1-Propanol | 3/5 | 55 | 96 |
| DMSO/1-Butanol | 3/5 | 55 | 83 |
| DMSO/1-Pentanol | 3/5 | 55 | 82 |
| DMSO/iso-Propanol | 3/5 | 55 | 89 |
| DMSO/iso-Butanol | 3/5 | 55 | 95 |
| DMSO/t-Butanol | 3/5 | 55 | 93 |
| DMSO/Ethoxy Ethanol | 3/5 | 55 | 69 |
| DMSO/Acetonitrile | 3/5 | 55 | 93 |
| DMSO/Acetone | 3/5 | 55 | 99 |
| DMSO/Methyl Ethyl Ketone | 3/5 | 55 | 85 |
| DMSO/MIBK | 3/5 | 55 | 94 |
| DMSO/Diethyl Ketone | 3/5 | 55 | 95 |
| DMSO/Ethyl Acetate | 3/5 | 55 | 97 |
| DMSO/iso-Propyl Acetate | 3/5 | 55 | 93 |
| DMSO/iso-Butyl Acetate | 3/5 | 55 | 96 |
| DMSO/n-Pentyl Acetate | 3/5 | 55 | 90 |
| DMSO/DCM | 3/5 | 55 | 80 |
| DMSO/1,4-Dioxane | 3/5 | 55 | 98 |
| DMSO/THF | 3/5 | 55 | 85 |
| DMSO/Toluene | 3/5 | 55 | 98 |
| DMSO/Xylene | 3/5 | 55 | 84 |

iii. Methoxy Ethanol Combinations:

TABLE 4

| | Amount | Temperature | Yield |
|---|---|---|---|
| Solvent/Anti-solvent | (vol) | (° C.) | (% w/w) |
| Methoxy Ethanol/Methanol | 8/5 | 115 | 85.5 |
| Methoxy Ethanol/1-Propanol | 8/5 | 115 | 88.4 |
| Methoxy Ethanol/1-Butanol | 8/5 | 115 | 76.5 |
| Methoxy Ethanol/1-Pentanol | 8/5 | 115 | 89.5 |
| Methoxy Ethanol/iso-Propanol | 8/5 | 115 | 84.0 |
| Methoxy Ethanol/iso-Butanol | 8/5 | 115 | 82.5 |
| Methoxy Ethanol/t-Butanol | 8/5 | 115 | 78.9 |
| Methoxy Ethanol/Ethoxy | 8/5 | 115 | 89.2 |
| Ethanol | | | |
| Methoxy Ethanol/Acetonitrile | 8/5 | 115 | 86.5 |
| Methoxy Ethanol/Acetone | 8/5 | 115 | 92.0 |
| Methoxy Ethanol/Ethyl | 8/5 | 115 | 92.3 |
| Acetate | | | |
| Methoxy Ethanol/iso-Propyl | 8/5 | 115 | 96.0 |
| Acetate | | | |
| Methoxy Ethanol/n-Pentyl | 8/5 | 115 | 93.0 |
| Acetate | | | |
| Methoxy Ethanol/DCM | 8/5 | 115 | 98.0 |
| Methoxy Ethanol/1,4-Dioxane | 8/5 | 115 | 95.0 |
| Methoxy Ethanol/THF | 8/5 | 115 | 89.2 |
| Methoxy Ethanol/TBME | 8/5 | 115 | 97.0 |
| Methoxy Ethanol/Toluene | 8/5 | 115 | 83.0 |
| Methoxy Ethanol/Xylene | 8/5 | 115 | 91.7 |
| Methoxy Ethanol/MIBK | 8/5 | 115 | 91.0 |
| Methoxy Ethanol/MEK | 8/5 | 115 | 86.0 |
| Methoxy Ethanol/Diethyl | 8/5 | 115 | 82.0 |
| Ether | | | |

### Form III

### A. Single Solvent: Solutions

TABLE 5

| | Amount | Temperature | Yield |
|---|---|---|---|
| Solvent | (vol) | (° C.) | (% w/w) |
| Ethyl Acetoacetate | 5 | 112 | 60 |

### B. Combination of Solvents: Solutions

TABLE 6

| | Amount | Temperature | Yield |
|---|---|---|---|
| Solvent/Anti-solvent | (vol) | (° C.) | (% w/w) |
| EAA/iso-Butyl Acetate | 5/5 | 112 | 35 |

### Form IV

### A. Single Solvent: Solutions

TABLE 7

| | Amount | Temperature | Yield |
|---|---|---|---|
| Solvent | (vol) | (° C.) | (% w/w) |
| Water | 5 | 62 | 66 |

### B. Combination of Solvents: Solutions

TABLE 8

| | Amount | Temperature | Yield |
|---|---|---|---|
| Solvent/Anti-solvent | (vol) | (° C.) | (% w/w) |
| Water/Methanol | 5/20 | 75 | 83 |
| Water/Ethanol | 5/20 | 75 | 89 |
| Water/1-Propanol | 5/20 | 75 | 81 |
| Water/iso-Propanol | 5/20 | 75 | 96 |
| Water/t-Butanol | 5/20 | 75 | 89 |
| Water/Ethoxy Ethanol | 5/20 | 75 | 87 |
| Water/Acetonitrile | 5/20 | 75 | 80 |
| Water/Acetone | 5/20 | 75 | 84 |
| Water/1,4-Dioxane | 5/20 | 75 | 93 |
| Water/THF | 5/30 | 75 | 51 |

The pharmaceutical composition according to the tenth aspect of the present invention can be a solution or suspension, but is preferably a solid oral dosage form. Preferred oral dosage forms in accordance with the invention include tablets, capsules and the like which, optionally, may be coated if desired. Tablets can be prepared by conventional techniques, including direct compression, wet granulation and dry granulation. Capsules are generally formed from a gelatine material and can include a conventionally prepared granulate of excipients in accordance with the invention.

The pharmaceutical composition according to the present invention typically comprises one or more conventional pharmaceutically acceptable excipient(s) selected from the group comprising a filler, a binder, a disintegrant, a lubricant and optionally further comprises at least one excipient selected from colouring agents, adsorbents, surfactants, film formers and plasticizers.

If the solid pharmaceutical formulation is in the form of coated tablets, the coating may be prepared from at least one film-former such as hydroxypropyl methyl cellulose, hydroxypropyl cellulose or methacrylate polymers which optionally may contain at least one plasticizer such as polyethylene glycols, dibutyl sebacate, triethyl citrate, and other pharmaceutical auxiliary substances conventional for film coatings, such as pigments, fillers and others.

Preferably, the pharmaceutical compositions according to the tenth aspect of the invention are for use in treating disorders related to abnormal protein kinase (PK) activity. Such diseases include, but are not limited to, diabetes, hepatic cirrhosis, cardiovascular disease such as atherosclerosis, angiogenesis, immunological disease such as autoimmune disease, malignant gastrointestinal stromal tumour (GIST) and metastatic renal cell carcinoma (MRCC).

The following paragraphs enumerated consecutively from 1 through 77 provide for various aspects of the present invention. In one embodiment, the present invention provides:

1. A crystalline form III of sunitinib malate characterized by an X-ray diffraction pattern having three or more peaks at 2θ values selected from 4.05, 8.02, 9.13, 10.44, 12.01, 16.00 and 17.80±0.2 °2θ.
2. A crystalline form III according to paragraph 1, characterized by a differential scanning calorimetry (DSC) with an endothermic peak at about 227° C.
3. A crystalline form III according to paragraph 1 or 2, which has a capillary melting point of about 216° C.
4. A crystalline form III according to any one of paragraphs 1 to 3, characterized by a thermo-gravimetric analysis (TGA) loss of about 0.29%.
5. A crystalline form III according to any one of paragraphs 1 to 4, which is non-hygroscopic and/or stable.
6. A process for the preparation of a crystalline form III of sunitinib malate according to any one of paragraphs 1 to 5, comprising the steps of:
(a) dissolving or suspending sunitinib malate, or sunitinib and malic acid, in a solvent;
(b) cooling the solution or suspension obtained in step (a);
(c) isolating the crystalline solid obtained in step (b); and
(d) drying the solid obtained in step (c).
7. A process according to paragraph 6, wherein in step (a) sunitinib malate is dissolved.
8. A process according to paragraph 6 or 7, wherein the solvent in step (a) is a non-hydroxylic solvent.
9. A process according to paragraph 8, wherein the non-hydroxylic solvent is an ester.
10. A process according to paragraph 9, wherein the ester is ethyl acetoacetate.
11. A process according to any one of paragraphs 6 to 10, wherein the solvent in step (a) is heated to dissolve the sunitinib malate.
12. A process according to paragraph 11, wherein solvent is heated at reflux temperature between 110-115° C.
13. A process according to any one of paragraphs 6 to 12, wherein step (b) comprises cooling to ambient temperature.
14. A process for the preparation of a crystalline form III of sunitinib malate according to any one of paragraphs 1 to 5, comprising the steps of:
(a) dissolving or suspending sunitinib malate, or sunitinib and malic acid, in a solvent;
(b) adding an anti-solvent to the solution or suspension obtained in step (a);
(c) cooling the solution or suspension obtained in step (b);
(d) isolating the crystalline solid obtained in step (c); and
(e) drying the solid obtained in step (d).
15. A process according to paragraph 14, wherein in step (a) sunitinib malate is dissolved.
16. A process according to paragraph 14 or 15, wherein the solvent in step (a) is a non-hydroxylic solvent.
17. A process according to paragraph 16, wherein the non-hydroxylic solvent is an ester.
18. A process according to paragraph 17, wherein the ester is ethyl acetoacetate.
19. A process according to any one of paragraphs 14 to 18, wherein the solvent in step (a) is heated at reflux temperature.
20. A process according to paragraph 19, wherein the reflux temperature is 110-115° C.
21. A process according to any one of paragraphs 14 to 20, wherein step (c) comprises cooling to ambient temperature.
22. A process according to any one of paragraphs 14 to 21, wherein the anti-solvent in step (b) is a non-hydroxylic solvent.
23. A process according to paragraph 22, wherein the non-hydroxylic solvent is an ester, a ketone or a hydrocarbon.
24. A process according to paragraph 23, wherein the non-hydroxylic solvent is an ester.
25. A process according to paragraph 24, wherein the ester is iso-butyl acetate.
26. A crystalline form IV of sunitinib malate characterized by an X-ray diffraction pattern having three or more peaks at 2θ values selected from 8.69, 13.01, 19.40, 20.32, 21.80, 24.18, 25.49, 26.13, 27.04, 28.23, 31.10 and 32.93±0.2 °2θ.
27. A crystalline form IV according to paragraph 26, characterized by a differential scanning calorimetry (DSC) with an endothermic peak at about 204° C.
28. A crystalline form IV according to paragraph 26 or 27, which has a capillary melting point of about 198° C.
29. A crystalline form IV according to any one of paragraphs 26 to 28, characterized by a thermo-gravimetric analysis (TGA) loss of about 0%.
30. A crystalline form IV according to any one of paragraphs 26 to 29, that is non-hygroscopic and/or stable.
31. A process for the preparation of a crystalline form IV of sunitinib malate according to any one of paragraphs 26 to 30, comprising the steps of:
(a) dissolving or suspending sunitinib malate, or sunitinib and malic acid, in a solvent;
(b) cooling the solution or suspension obtained in step (a);
(c) isolating the crystalline solid obtained in step (b); and
(d) drying the solid obtained in step (c).
32. A process according to paragraph 31, wherein in step (a) sunitinib malate is dissolved.
33. A process according to paragraph 31 or 32, wherein the solvent in step (a) is water.
34. A process according to any one of paragraphs 31 to 33, wherein the solvent in step (a) is heated.
35. A process according to paragraph 34, wherein the solvent in step (a) is heated at 60-80° C.
36. A process according to paragraph 35, wherein the solvent in step (a) is heated at about 62° C.
37. A process according to any one of paragraphs 31 to 36, wherein step (b) comprises cooling to ambient temperature.
38. A process for the preparation of a crystalline form IV of sunitinib malate according to any one of paragraphs 26 to 30, comprising the steps of:
(a) dissolving or suspending sunitinib malate, or sunitinib and malic acid, in a solvent;
(b) adding an anti-solvent to the solution or suspension obtained in step (a);
(c) cooling the solution or suspension obtained in step (b);
(d) isolating the crystalline solid obtained in step (c); and
(e) drying the solid obtained in step (d).
39. A process according to paragraph 38, wherein in step (a) sunitinib malate is dissolved.
40. A process according to paragraph 38 or 39, wherein the solvent in step (a) is water.
41. A process according to any one of paragraphs 38 to 40, wherein the solvent in step (a) is heated.
42. A process according to paragraph 41, wherein the solvent in step (a) is heated at 60-80° C.
43. A process according to paragraph 42, wherein the solvent in step (a) is heated at about 75° C.
44. A process according to any one of paragraphs 38 to 43, wherein step (c) comprises cooling to ambient temperature.
45. A process according to any one of paragraphs 38 to 44, wherein the anti-solvent is selected from an alcohol, a ketone, an ester, a nitrile, an ether, a hydrocarbon or a halogenated hydrocarbon.
46. A process according to paragraph 45, wherein the anti-solvent is selected from an alcohol, acetonitrile, acetone, 1,4-dioxane or THF.
47. A process according to paragraph 46, wherein the anti-solvent is an alcohol.
48. A process according to paragraph 47, wherein the anti-solvent is a C1 to C6 alcohol or a substituted alcohol.
49. A process according to paragraph 48, wherein the substituted alcohol is ethoxy ethanol.
50. A process according to paragraph 48, wherein the C1 to C6 alcohol is selected from methanol, ethanol, n-propanol, iso-propanol or t-butanol.
51. A process for the preparation of a crystalline form I of sunitinib malate, comprising the steps of:
(a) dissolving or suspending sunitinib malate, or sunitinib and malic acid, in a solvent;
(b) cooling the solution or suspension obtained in step (a);
(c) isolating the crystalline solid obtained in step (b); and
(d) drying the solid obtained in step (c).
52. A process according to paragraph 51, wherein in step (a) sunitinib malate is dissolved.
53. A process according to paragraph 51 or 52, wherein the solvent in step (a) is a hydroxylic solvent or a polar aprotic solvent.
54. A process according to paragraph 53, wherein the solvent in step (a) is selected from cyclopentanol, cyclohexanol, methoxy ethanol or N,N-dimethylacetamide.
55. A process according to any one of paragraphs 51 to 54, wherein the solvent in step (a) is heated to dissolve the sunitinib malate.
56. A process according to any one of paragraphs 51 to 55, wherein step (b) comprises cooling to ambient temperature.
57. A process for the preparation of a crystalline form I of sunitinib malate, comprising the steps of:
(a) dissolving or suspending sunitinib malate, or sunitinib and malic acid, in a solvent;
(b) adding an anti-solvent to the solution or suspension obtained in step (a);
(c) cooling the solution or suspension obtained in step (b);
(d) isolating the crystalline solid obtained in step (c); and
(e) drying the solid obtained in step (d).
58. A process according to paragraph 57, wherein in step (a) sunitinib malate is dissolved.
59. A process according to paragraph 57 or 58, wherein the solvent in step (a) is a polar aprotic solvent, an alcohol or an alkoxy alcohol.
60. A process according to paragraph 59, wherein the polar aprotic solvent is DMF, DMAc or DMSO.
61. A process according to paragraph 59, wherein the alkoxy alcohol is methoxy ethanol.
62. A process according to any one of paragraphs 57 to 61, wherein the solvent in step (a) is heated to dissolve the sunitinib malate.
63. A process according to paragraph 62, wherein is the solvent is heated between 55-115° C.
64. A process according to any one of paragraphs 57 to 63, wherein the anti-solvent is selected from an alcohol, a ketone, an ester, a nitrile, an ether, a hydrocarbon or a halogenated hydrocarbon.
65. A process according to paragraph 64, wherein the anti-solvent is selected from water, methanol, ethanol, 1-propanol, 1-butanol, 1-pentanol, iso-propanol, iso-butanol, t-butanol, ethoxy ethanol, acetonitrile, acetone, methyl ethyl ketone, methyl iso-butyl ketone, diethyl ketone, ethyl acetate, iso-propyl acetate, iso-butyl acetate, n-pentyl acetate, DCM, 1,4-dioxane, THF, t-butyl methyl ether, diethyl ether, toluene or xylene.
66. A process according to any one of paragraphs 57 to 65, wherein step (c) comprises cooling to ambient temperature.
67. A crystalline form I of sunitinib malate obtained by a process according to any one of paragraphs 51 to 66.
68. A pharmaceutical composition comprising sunitinib malate form III according to any one of paragraphs 1 to 5, or sunitinib malate form IV according to any one of paragraphs 26 to 30, or sunitinib malate form I according to paragraph 67.
69. A pharmaceutical composition according to paragraph 68, for treating or preventing cancer or a tumour.
70. A pharmaceutical composition according to paragraph 69, for treating or preventing unresectable and/or metastatic malignant gastrointestinal stromal tumour (GIST) or advanced and/or metastatic renal cell carcinoma (MRCC).
71. Sunitinib malate form III according to any one of paragraphs 1 to 5, or sunitinib malate form IV according to any one of paragraphs 26 to 30, or sunitinib malate form I according to paragraph 67, for use in medicine.
72. Sunitinib malate according to paragraph 71, for treating or preventing cancer or a tumour.
73. Sunitinib malate according to paragraph 72, for treating or preventing unresectable and/or metastatic malignant gastrointestinal stromal tumour (GIST) or advanced and/or metastatic renal cell carcinoma (MRCC).
74. Use of sunitinib malate form III according to any one of paragraphs 1 to 5, or sunitinib malate form IV according to any one of paragraphs 26 to 30, or sunitinib malate form I according to paragraph 67, in the manufacture of a medicament for treating or preventing cancer or a tumour.
75. Use of sunitinib malate form III according to any one of paragraphs 1 to 5, or sunitinib malate form IV according to any one of paragraphs 26 to 30, or sunitinib malate form I according to paragraph 67, in the manufacture of a medicament for treating or preventing unresectable and/or metastatic malignant gastrointestinal stromal tumour (GIST) or advanced and/or metastatic renal cell carcinoma (MRCC).
76. A method of treating or preventing cancer or a tumour, the method comprising administering to a patient in need thereof a therapeutically of prophylactically effective amount of sunitinib malate form III according to any one of paragraphs 1 to 5, or sunitinib malate form IV according to any one of paragraphs 26 to 30, or sunitinib malate form I according to paragraph 67.
77. A method of treating or preventing unresectable and/or metastatic malignant gastrointestinal stromal tumour (GIST) or advanced and/or metastatic renal cell carcinoma (MRCC), the method comprising administering to a patient in need thereof a therapeutically of prophylactically effective amount of sunitinib malate form III according to any one of paragraphs 1 to 5, or sunitinib malate form IV according to any one of paragraphs 26 to 30, or sunitinib malate form I according to paragraph 67.
The details of the invention, its objects and advantages are illustrated below in greater detail by non-limiting examples.

### EXAMPLES

### Example 1 (Form III) (see Table 5)

Sunitinib malate (1 eq) was charged in ethyl acetoacetate (5-vol) in a two-neck round-bottom flask equipped with a thermopocket and a reflux condenser and was stirred at 23-27° C. for 10 minutes. A slurry was observed which was heated to 110-115° C. and then maintained at this temperature for about 15-20 minutes. A clear solution was observed. The reaction mixture was allowed to cool to 23-27° C. gradually over a period of 1-2 hours and stirred at this temperature for about 15-20 minutes. A slurry was observed. The solid was filtered on a Buchner funnel under vacuum and dried on a rotavapour at 40° C. at high vacuum to obtain a yellow solid, which was characterized as sunitinib malate form III. Yield=60%.

### Example 2 (Form III) (see Table 6)

Sunitinib malate (1 eq) was charged in ethyl acetoacetate (5-vol) in a two-neck round-bottom flask equipped with a thermopocket and a reflux condenser and was stirred at 23-27° C. for 10 minutes. A slurry was observed which was heated to about 112° C. and then maintained at this temperature for about 15-20 minutes. A clear solution was observed. Iso-butyl acetate (5-vol) was added and the reaction mixture was stirred for a further 15-20 minutes at about 112° C. The reaction mixture was allowed to cool to 23-27° C. gradually over a period of 1-2 hours and stirred at this temperature for about 15-20 minutes. A solid was observed. The solid was filtered on a Buchner funnel under vacuum and dried on a rotavapour at 40° C. at high vacuum to obtain a yellow solid, which was characterized as sunitinib malate form III. Yield=35%.

### Example 3 (Form IV) (see Table 7)

Sunitinib malate (1 eq) was charged in water (5-vol) in a two-neck round-bottom flask equipped with a thermopocket and a reflux condenser and was stirred at 23-27° C. for 10 minutes. A slurry was observed. The reaction mixture was heated to about 62° C. and maintained at this temperature for about 15-20 minutes. A clear solution was observed. The reaction mixture was allowed to cool to 23-27° C. gradually over a period of 45-60 minutes and stirred at this temperature for 30 minutes. A slurry was observed. The solid was filtered on a Buchner funnel under vacuum and dried on a rotavapour at 40° C. under vacuum to obtain a yellow solid, which was characterized as sunitinib malate form IV. Yield=66%.

### Example 4 (Form IV) (see Table 8)

Sunitinib malate (1 eq) was charged in water (5-vol) in a two-neck round-bottom flask equipped with a thermopocket and a reflux condenser and was stirred at 23-27° C. for 10 minutes. A slurry was observed. The reaction mixture was heated to about 75° C. and maintained at this temperature for about 15-20 minutes. A clear solution was observed. Anti-solvent* (a-j)(20-30 vol) was added at about 75° C. and the reaction mixture stirred at this temperature for a further 15-20 minutes. The reaction mixture was allowed to cool to 23-27° C. gradually over a period of 45-60 minutes and stirred at this temperature for 30 minutes. A solid was observed. The solid was filtered on a Buchner funnel under vacuum and dried on a rotavapour at 40° C. under vacuum to obtain a yellow solid, which was characterized as sunitinib malate form IV. Yield=51-96%.

*The anti-solvent was selected from one or more of the following: a. Methanol, b. Ethanol, c. 1-Propanol, d. iso-Propanol, e. t-Butanol, f. Ethoxy Ethanol, g. Acetonitrile, h. Acetone, i. 1,4-Dioxane, j. THF.

### Example 5 (Form I) (see Table 1)

Cyclopentanol (25 vol) and sunitinib malate (1 eq) were charged to a two-neck round-bottom flask equipped with a thermopocket and a reflux condenser and stirred at 23-27° C. for 10 minutes. A slurry was observed. The reaction mixture was heated to about 100° C. and then maintained at this temperature for about 15-20 minutes. A clear solution was observed. The reaction mixture was allowed to cool to 23-27° C. gradually over a period of 45-60 minutes and stirred at this temperature for 30 minutes. A solid was observed. The solid was filtered on a Buchner funnel under vacuum and dried on a rotavapour at 40° C. under high vacuum to obtain a yellow solid, which was characterized as sunitinib malate form I. Yield=69%.

### Example 6 (Form I) (see Table 1)

Cyclohexanol (25-vol) and sunitinib malate (1 eq) were charged to a two-neck round-bottom flask equipped with a thermopocket and a reflux condenser and stirred at 23-27° C. for 10 minutes. A slurry was observed. The reaction mixture was heated to about 110° C. and then maintained at this temperature for about 15-20 minutes. A clear solution was observed. The reaction mixture was allowed to cool to 23-27° C. gradually over a period of 45-60 minutes and stirred at this temperature for 30 minutes. A solid was observed. The solid was filtered on a Buchner funnel under vacuum and dried on a rotavapour at 40° C. under high vacuum to obtain a yellow solid, which was characterized as sunitinib malate form I. Yield=98%.

### Example 7 (Form I) (see Table 1)

Methoxy ethanol (25 vol) and sunitinib malate (1 eq) were charged to a two-neck round-bottom flask equipped with a thermopocket and a reflux condenser and stirred at 23-27° C. for 10 minutes. A slurry was observed. The reaction mixture was heated to about 122° C. and then maintained at this temperature for about 15-20 minutes. A clear solution was observed. The reaction mixture was allowed to cool to 23-27° C. gradually over a period of 45-60 minutes and stirred at this temperature for 30 minutes. A solid was observed. The solid was filtered on a Buchner funnel under vacuum and dried on a rotavapour at 40° C. under high vacuum to obtain a yellow solid, which was characterized as sunitinib malate form I. Yield=60%.

### Example 8 (Form I) (see Table 1)

N,N-Dimethylacetamide (25 vol) and sunitinib malate (1 eq) were charged to a two-neck round-bottom flask equipped with a thermopocket and a reflux condenser and stirred at 23-27° C. for 10 minutes. A slurry was observed. The reaction mixture was heated to about 99° C. and then maintained at this temperature for about 15-20 minutes. A clear solution was observed. The reaction mixture was allowed to cool to 23-27° C. gradually over a period of 45-60 minutes and stirred at this temperature for 30 minutes. A solid was observed. The solid was filtered on a Buchner funnel under vacuum and dried on a rotavapour at 40° C. under high vacuum to obtain a yellow solid, which was characterized as sunitinib malate form I. Yield=77%.

### Example 9 (Form I) (see Table 2)

DMF (3-vol) and sunitinib malate (1 eq) were charged to a two-neck round-bottom flask equipped with a thermopocket and a reflux condenser and stirred at 23-27° C. for 10 minutes. A slurry was observed. The reaction mixture was heated to about 80° C. and then maintained at this temperature for about 5-10 minutes. A clear solution was observed. Anti-solvent* (a-z) (5-10 vol) was added and the reaction mixture was stirred at about 80° C. for a further 15-20 minutes. The reaction mixture was allowed to cool to 23-27° C. gradually over a period of 45-60 minutes and stirred at this temperature for 30 minutes. A solid was observed. The solid was filtered on a Buchner funnel under vacuum and dried on a rotavapour at 40° C. under high vacuum to obtain a yellow solid, which was characterized as sunitinib malate form I. Yield=75-99%.

*The anti-solvent was selected from one or more of the following: a. Water, b. Methanol, c. Ethanol, d. 1-Propanol, e. 1-Butanol, f. 1-Pentanol, g. iso-Propanol, h. iso-Butanol, i. t-Butanol, j. Ethoxy Ethanol, k. Acetonitrile, 1. Acetone, m. Methyl Ethyl Ketone, n. Methyl iso-Butyl Ketone, o. Diethyl Ketone, p. Ethyl Acetate, q. iso-Propyl Acetate, r. iso-Butyl Acetate, s. n-Pentyl Acetate, t. DCM, u. 1,4-Dioxane, v. THF, w. t-Butyl Methyl Ether, x. Diethyl Ether, y. Toluene, z. Xylene.

### Example 10 (Form I) (see Table 3)

DMSO (3-vol) and sunitinib malate (1 eq) were charged to a two-neck round-bottom flask equipped with a thermopocket and a reflux condenser and stirred at 23-27° C. for 10 minutes. A slurry was observed. The reaction mixture was heated to about 55° C. and then maintained at this temperature for about 5-10 minutes. A clear solution was observed. Anti-solvent* (a-x) (5-30 vol) was added and the reaction mixture was stirred at about 55° C. for a further 15-20 minutes. The reaction mixture was allowed to cool to 23-27° C. gradually over a period of 45-60 minutes and stirred at this temperature for 30 minutes. A solid was observed. The solid was filtered on a Buchner funnel under vacuum and dried on a rotavapour at 40° C. under high vacuum to obtain a yellow solid, which was characterized as sunitinib malate form I. Yield=69-99%.

*The anti-solvent was selected from one or more of the following: a. Water, b. Methanol, c. Ethanol, d. 1-Propanol, e. 1-Butanol, f. 1-Pentanol, g. iso-Propanol, h. iso-Butanol, i. t-Butanol, j. Ethoxy Ethanol, k. Acetonitrile, 1. Acetone, m. Methyl Ethyl Ketone, n. Methyl iso-Butyl Ketone, o. Diethyl Ketone, p. Ethyl Acetate, q. iso-Propyl Acetate, r. iso-Butyl Acetate, s. n-Pentyl Acetate, t. DCM, u. 1,4-Dioxane, v. THF, w. Toluene, x. Xylene.

### Example 11 (Form I) (see Table 4)

Methoxy ethanol (8-vol) and sunitinib malate (1 eq) were charged to a two-neck round-bottom flask equipped with a thermopocket and a reflux condenser and stirred at 23-27° C. for 10 minutes. A slurry was observed. The reaction mixture was heated to about 115° C. and then maintained at this temperature for about 15-20 minutes. A clear solution was observed. Anti-solvent* (a-v) (5-vol) was added and the reaction mixture was stirred at about 115° C. for a further 15-20 minutes. The reaction mixture was allowed to cool to 23-27° C. gradually over a period of 45-60 minutes and stirred at this temperature for 30 minutes. A solid was observed. The solid was filtered on a Buchner funnel under vacuum and dried on a rotavapour at 40° C. under high vacuum to obtain a yellow solid, which was characterized as sunitinib malate form I. Yield=76-98%.

*The anti-solvent was selected from one or more of the following: a. Methanol, b. 1-Propanol, c. 1-Butanol, d. 1-Pentanol, e. iso-Propanol, f. iso-Butanol, g. t-Butanol, h. Ethoxy Ethanol, i. Acetonitrile, j. Acetone, k. Ethyl Acetate, 1. iso-Propyl Acetate, m. n-Pentyl Acetate, n. DCM, o. 1,4-Dioxane, p. THF, q. t-Butyl Methyl Ether, r. Toluene, s. Xylene, t. Methyl iso-Butyl Ketone, u. Methyl Ethyl Ketone, v. Diethyl Ether.

1-77. (canceled)

78. A crystalline form III of sunitinib malate characterized by an X-ray diffraction pattern having three or more peaks at 2θ values selected from 4.05, 8.02, 9.13, 10.44, 12.01, 16.00 and 17.80±0.2 °2θ.

79. A crystalline form III according to claim 78:
(i) characterized by a differential scanning calorimetry (DSC) with an endothermic peak at about 227° C.; and/or
(ii) which has a capillary melting point of about 216° C.; and/or
(iii) characterized by a thermo-gravimetric analysis (TGA) loss of about 0.29%; and/or
(iv) which is non-hygroscopic and/or stable.

80. A process for the preparation of a crystalline form III of sunitinib malate according to claim 78, comprising the steps of:
(a) dissolving or suspending sunitinib malate, or sunitinib and malic acid, in a solvent;
(b) cooling the solution or suspension obtained in step (a);
(c) isolating the crystalline solid obtained in step (b); and
(d) drying the solid obtained in step (c).

81. A process according to claim 80, wherein:
(i) in step (a) sunitinib malate is dissolved; and/or
(ii) step (b) comprises cooling to ambient temperature.

82. A process according to claim 80, wherein the solvent in step (a) is:
(i) a non-hydroxylic solvent; and/or
(ii) an ester; and/or
(iii) ethyl acetoacetate; and/or
(iv) heated to dissolve the sunitinib malate; and/or
(v) heated at reflux temperature between 110-115° C. to dissolve the sunitinib malate.

83. A process for the preparation of a crystalline form III of sunitinib malate according to claim 78, comprising the steps of:
(a) dissolving or suspending sunitinib malate, or sunitinib and malic acid, in a solvent;
(b) adding an anti-solvent to the solution or suspension obtained in step (a);
(c) cooling the solution or suspension obtained in step (b);
(d) isolating the crystalline solid obtained in step (c); and
(e) drying the solid obtained in step (d).

84. A process according to claim 83, wherein:
(i) in step (a) sunitinib malate is dissolved; and/or
(ii) step (c) comprises cooling to ambient temperature.

85. A process according to claim 83, wherein the solvent in step (a) is:
(i) a non-hydroxylic solvent; and/or
(ii) an ester; and/or
(iii) ethyl acetoacetate; and/or
(iv) heated at reflux temperature; and/or
(v) heated at reflux temperature between 110-115° C.

86. A process according to claim 83, wherein the anti-solvent in step (b) is:
(i) a non-hydroxylic solvent; and/or
(ii) an ester, a ketone or a hydrocarbon; and/or
(iii) an ester; and/or
(iv) iso-butyl acetate.

87. A crystalline form IV of sunitinib malate characterized by an X-ray diffraction pattern having three or more peaks at 2θ values selected from 8.69, 13.01, 19.40, 20.32, 21.80, 24.18, 25.49, 26.13, 27.04, 28.23, 31.10 and 32.93±0.2 °2θ.

88. A crystalline form IV according to claim 87:
(i) characterized by a differential scanning calorimetry (DSC) with an endothermic peak at about 204° C.; and/or
(ii) which has a capillary melting point of about 198° C.; and/or
(iii) characterized by a thermo-gravimetric analysis (TGA) loss of about 0%; and/or
(iv) that is non-hygroscopic and/or stable.

89. A process for the preparation of a crystalline form IV of sunitinib malate according to claim 87, comprising the steps of:
(a) dissolving or suspending sunitinib malate, or sunitinib and malic acid, in a solvent;
(b) cooling the solution or suspension obtained in step (a);
(c) isolating the crystalline solid obtained in step (b); and
(d) drying the solid obtained in step (c).

90. A process according to claim 89, wherein:
(i) in step (a) sunitinib malate is dissolved; and/or
(ii) step (b) comprises cooling to ambient temperature.

91. A process according to claim 89, wherein the solvent in step (a) is:
(i) water; and/or
(ii) heated; and/or
(iii) heated at 60-80° C.; and/or
(iv) heated at about 62° C.

92. A process for the preparation of a crystalline form IV of sunitinib malate according to claim 87, comprising the steps of:
(a) dissolving or suspending sunitinib malate, or sunitinib and malic acid, in a solvent;
(b) adding an anti-solvent to the solution or suspension obtained in step (a);
(c) cooling the solution or suspension obtained in step (b);
(d) isolating the crystalline solid obtained in step (c); and
(e) drying the solid obtained in step (d).

93. A process according to claim 92, wherein:
(i) in step (a) sunitinib malate is dissolved; and/or
(ii) step (c) comprises cooling to ambient temperature.

94. A process according to claim 92, wherein the solvent in step (a) is:
(i) water; and/or
(ii) heated; and/or
(iii) heated at 60-80° C.; and/or
(iv) heated at about 75° C.

95. A process according to claim 92, wherein the anti-solvent is:
(i) selected from an alcohol, a ketone, an ester, a nitrile, an ether, a hydrocarbon or a halogenated hydrocarbon; and/or
(ii) selected from an alcohol, acetonitrile, acetone, 1,4-dioxane or THF; and/or
(iii) an alcohol; and/or
(iv) a C1 to C6 alcohol or a substituted alcohol; and/or
(v) ethoxy ethanol; and/or
(vi) selected from methanol, ethanol, n-propanol, iso-propanol or t-butanol.

96. A process for the preparation of a crystalline form I of sunitinib malate, comprising the steps of:
(a) dissolving or suspending sunitinib malate, or sunitinib and malic acid, in a solvent;
(b) cooling the solution or suspension obtained in step (a);
(c) isolating the crystalline solid obtained in step (b); and
(d) drying the solid obtained in step (c).

97. A process according to claim 96, wherein:
(i) in step (a) sunitinib malate is dissolved; and/or
(ii) step (b) comprises cooling to ambient temperature.

98. A process according to claim 96, wherein the solvent in step (a) is:
(i) a hydroxylic solvent or a polar aprotic solvent; and/or
(ii) selected from cyclopentanol, cyclohexanol, methoxy ethanol or N,N-dimethylacetamide; and/or
(iii) heated to dissolve the sunitinib malate.

99. A process for the preparation of a crystalline form I of sunitinib malate, comprising the steps of:
(a) dissolving or suspending sunitinib malate, or sunitinib and malic acid, in a solvent;
(b) adding an anti-solvent to the solution or suspension obtained in step (a);
(c) cooling the solution or suspension obtained in step (b);
(d) isolating the crystalline solid obtained in step (c); and
(e) drying the solid obtained in step (d).

100. A process according to claim 99, wherein:
(i) in step (a) sunitinib malate is dissolved; and/or
(ii) step (c) comprises cooling to ambient temperature.

101. A process according to claim 99, wherein the solvent in step (a) is:
(i) a polar aprotic solvent, an alcohol or an alkoxy alcohol; and/or
(ii) DMF, DMAc or DMSO; and/or
(iii) methoxy ethanol; and/or
(iv) heated to dissolve the sunitinib malate; and/or
(v) heated between 55-115° C.

102. A process according to claim 99, wherein the anti-solvent is:
(i) selected from an alcohol, a ketone, an ester, a nitrile, an ether, a hydrocarbon or a halogenated hydrocarbon; and/or
(ii) selected from water, methanol, ethanol, 1-propanol, 1-butanol, 1-pentanol, iso-propanol, iso-butanol, t-butanol, ethoxy ethanol, acetonitrile, acetone, methyl ethyl ketone, methyl iso-butyl ketone, diethyl ketone, ethyl acetate, iso-propyl acetate, iso-butyl acetate, n-pentyl acetate, DCM, 1,4-dioxane, THF, t-butyl methyl ether, diethyl ether, toluene or xylene.

103. A crystalline form I of sunitinib malate obtained by a process according to claim 96.

104. A crystalline form I of sunitinib malate obtained by a process according to claim 99.

105. A pharmaceutical composition comprising sunitinib malate form III according to claim 78.

106. A pharmaceutical composition according to claim 105, for:
(i) treating or preventing cancer or a tumour; and/or
(ii) treating or preventing unresectable and/or metastatic malignant gastrointestinal stromal tumour (GIST) or advanced and/or metastatic renal cell carcinoma (MRCC).

107. A pharmaceutical composition comprising sunitinib malate form IV according to claim 87.

108. A pharmaceutical composition according to claim 107, for:
(i) treating or preventing cancer or a tumour; and/or
(ii) treating or preventing unresectable and/or metastatic malignant gastrointestinal stromal tumour (GIST) or advanced and/or metastatic renal cell carcinoma (MRCC).

109. A pharmaceutical composition comprising sunitinib malate form I according to claim 103.

110. A pharmaceutical composition according to claim 109, for:
(i) treating or preventing cancer or a tumour; and/or
(ii) treating or preventing unresectable and/or metastatic malignant gastrointestinal stromal tumour (GIST) or advanced and/or metastatic renal cell carcinoma (MRCC).

111. A pharmaceutical composition comprising sunitinib malate form I according to claim 104.

112. A pharmaceutical composition according to claim 111, for:
(i) treating or preventing cancer or a tumour; and/or
(ii) treating or preventing unresectable and/or metastatic malignant gastrointestinal stromal tumour (GIST) or advanced and/or metastatic renal cell carcinoma (MRCC).

113. A method of treating or preventing cancer or a tumour or unresectable and/or metastatic malignant gastrointestinal stromal tumour (GIST) or advanced and/or metastatic renal cell carcinoma (MRCC), the method comprising administering to a patient in need thereof a therapeutically of prophylactically effective amount of sunitinib malate form III according to claim 78.

114. A method of treating or preventing cancer or a tumour or unresectable and/or metastatic malignant gastrointestinal stromal tumour (GIST) or advanced and/or metastatic renal cell carcinoma (MRCC), the method comprising administering to a patient in need thereof a therapeutically of prophylactically effective amount of sunitinib malate form IV according to claim 87.

115. A method of treating or preventing cancer or a tumour or unresectable and/or metastatic malignant gastrointestinal stromal tumour (GIST) or advanced and/or metastatic renal cell carcinoma (MRCC), the method comprising administering to a patient in need thereof a therapeutically of prophylactically effective amount of sunitinib malate form I according to claim 103.

116. A method of treating or preventing cancer or a tumour or unresectable and/or metastatic malignant gastrointestinal stromal tumour (GIST) or advanced and/or metastatic renal cell carcinoma (MRCC), the method comprising administering to a patient in need thereof a therapeutically of prophylactically effective amount of sunitinib malate form I according to claim 104.
